(19) United States
(12) Patent Application Publication
(10) Pub. No.: US 2019/0272921 A1
(43) Pub. Date: Sep. 5, 2019

(54) Automated Diagnostic Support System for Clinical Documentation Workflows

(71) Applicant: MModal IP LLC, Franklin, TN (US)

(72) Inventor: Detlef Koll, Pittsburgh, PA (US)

(21) Appl. No.: 16/290,042

(22) Filed: Mar. 1, 2019

Related U.S. Application Data
(60) Provisional application No. 62/637,463, filed on Mar. 2, 2018.

(51) Int. Cl. G16H 50/20; G10L 15/26; G10L 15/18; G16H 30/20

(52) U.S. Cl. CPC G16H 50/20; G16H 30/20; G10L 15/1815; G10L 15/265

(57) ABSTRACT

A computer system automatically generates a first diagnosis of a patient based on input such as one or more medical images. The computer system receives input representing diagnostic intent from a human user. The system determines whether to provide the first diagnosis to the human user based on the first diagnosis and the diagnostic intent, such as by determining whether the first diagnosis and the diagnostic intent agree with each other, and only providing the first diagnosis to the human user if the first diagnosis disagrees with the diagnostic intent of the human user.

### BACKGROUND

To diagnose a patient, traditionally a physician examines the patient and then uses his or her own expert professional knowledge and judgment to produce and document a diagnosis for the patient. More recently, various Computer Aided Diagnostic (CAD) systems have been created to automate the generation of a clinical diagnosis based on current and past clinical and social information relating to the patient. In particular, deep learning systems are increasingly used to automatically analyze a radiology image (e.g., an ultrasound, CT, or MRI image) and to automatically detect abnormalities in the image, and even to derive a full clinical diagnosis from the image. Existing CAD systems can perform at or above the level of humans in certain narrow areas of use, such as reading sensitivity (recall) and specificity (precision). In most other ways, however, existing CAD systems do not perform as well as human experts. Furthermore, providing a system that meets or exceeds human accuracy in all settings is a difficult and long process.

### SUMMARY

A computer system automatically generates a first diagnosis of a patient based on input such as one or more medical images. The computer system receives input representing diagnostic intent from a human user. The system determines whether to provide the first diagnosis to the human user based on the first diagnosis and the diagnostic intent, such as by determining whether the first diagnosis and the diagnostic intent agree with each other, and only providing the first diagnosis to the human user if the first diagnosis disagrees with the diagnostic intent of the human user.

Other features and advantages of various aspects and embodiments of the present invention will become apparent from the following description and from the claims.

### BRIEF DESCRIPTION OF THE DRAWINGS

FIG. 1 is a dataflow diagram of a computer system for automatically providing output of a CAD system to a physician only when doing so provides a benefit to the physician according to one embodiment of the present invention.

FIG. 2 is a flowchart of a method performed by the system of FIG. 1 according to one embodiment of the present invention.

### DETAILED DESCRIPTION

There is emerging evidence that even if a Computer Aided Diagnostic (CAD) system does not perform as well as a human in terms of accuracy, a combination of the CAD system and a human expert may exceed the individual accuracy of either. This general effect is known in the machine learning community as “boosting,” which refers to a technique in which an ensemble of multiple weak classifiers is combined to make a strong classifier. In the case of clinical diagnosis, however, the physician is responsible for the final diagnosis and cannot, therefore, be treated merely as a “weak classifier” that can be overridden by other classifiers (such as the CAD system). Instead, all input from other weaker classifiers, such as CAD systems, must be evaluated and consolidated by the physician into a final diagnosis, which can require a significant amount of valuable physician time. As a result of this aspect of clinical diagnosis, simple existing boosting techniques cannot be applied to the problem of clinical diagnosis. One problem posed by the prior art, therefore, is how to leverage the benefits of CAD systems even when they are not perfectly accurate, and in light of the requirement that the physician approve of the final diagnosis, while minimizing the amount of physician time and effort required to produce the final diagnosis. From a technical perspective, one problem posed by the prior art is how to develop a computer-implemented system that can use a CAD system to automatically generate a diagnosis of a patient, and which can automatically tailor the output of the CAD system to based on an automated comparison between a diagnosis generated automatically by the CAD system and an indication of diagnostic intent received from the patient's physician.

In a simplified example, assume that in a certain use case the physician's accuracy is 95% and that the CAD system's accuracy is 93%. (Although the description herein refers solely to accuracy for ease of explanation, in practice such accuracy may be divided into and measured by reference to both specificity and sensitivity.) If the physician's and CAD system's errors are perfectly correlated (i.e., if the physician's errors are a subset of the CAD system's errors), then the CAD system would have no value in improving the diagnostic accuracy of the physician. In practice, however, it is not usually the case that the physician's and CAD system's errors are perfectly correlated.

Therefore, solely for purposes of example and without limitation, assume instead that:
on 3% of the cases, both the physician and the CAD system make the same mistake (misclassification);
on 2% of the cases, the physician makes mistakes and the CAD system is accurate;
on 4% of the cases, the physician is accurate and the CAD system is inaccurate.

As described above, existing CAD systems typically always display the CAD-generated diagnosis to the physician for review. Naively displaying all of the CAD-generated diagnoses to the physician in the scenario described above would result in the following:
91% of all cases would be correctly classified by both the physician and the CAD system. Displaying the output of the CAD system to the physician in these cases, however, does not provide any useful information to the physician, because the physician has already classified these cases correctly and does not need to review the output of the CAD system to improve the diagnosis that was generated manually by the physician. As a result, displaying the output of the CAD system to the physician in these cases results in wasting the physician's time and therefore reduces the overall efficiency of the human-computer system.
3% of all cases would be misclassified by both the physician and the CAD system. Displaying the output of the CAD system to the physician in these cases does not provide any useful information to the physician, because even though the physician has misclassified these cases, reviewing the CAD system's misclassified diagnoses will not help the physician to fix the misclassification in the physician's manually-generated diagnosis. As a result, displaying the output of the CAD system to the physician in these cases results in wasting the physician's time and therefore reduces the overall efficiency of the human-computer system.
4% of all cases would be classified correctly by the physician and classified incorrectly by the CAD system. Displaying the output of the CAD system to the physician in these cases is not only a waste of the physician's time; it is also affirmatively harmful to the physician because this might reduce the physician's trust in the system.
2% of all cases would be classified correctly by the CAD system and classified incorrectly by the physician. Providing the CAD system's automatically-generated diagnoses in these cases to the physician would be useful, because the physician might correct his or her incorrect classifications to correct classifications based on the CAD system's output. As a result, displaying the output of the CAD system to the physician in these cases may result in modifying the output of the CAD system to make it more accurate, and thereby result in an improvement in the accuracy of the human-computer system.

As the above example illustrates, one problem with existing systems is that, by providing the CAD system's output to the physician in all cases, they make highly inefficient use of the physician's time by providing the physician with the CAD system's output to review even in cases in which such review at best provides no benefit, and at worst is affirmatively disadvantageous. In the particular example above, in which only 2% of cases involve CAD output which is useful for the physician to review, the amount of physician time wasted is very significant.

As the above example further illustrates, another problem with existing systems is that, by providing the CAD system's output to the physician in all cases, they involve the CAD system generating and providing output in cases in which doing so is not likely to result in any improvement in accuracy or efficiency of the CAD system. As a result, such CAD systems perform generate and provide output which not only is unlikely to improvement the accuracy or efficiency of the CAD system, but which may actually result in a decrease in accuracy of the CAD system (if, for example, the physician modifies the CAD system's initial output to make it less accurate) and a decrease in efficiency of the CAD system (by, for example, causing the CAD system to generate and provide output which does not result in any increase in accuracy of the results produced by the CAD system).

Embodiments of the present invention address these and other problems of prior art systems by automatically and selectively providing CAD system output to a physician only in cases in which embodiments of the present invention determine that it would be valuable to provide such output to the physician. In other cases, embodiments of the present invention suppress or otherwise do not provide such output to the physician. As a result, the efficiency of the overall system (including both the CAD system and the physician) at generating accurate diagnoses is improved.

More specifically, the accuracy and efficiency of the CAD system itself is improved by embodiments of the present invention, relative to the prior art. For example, embodiments of the present invention solve the above-mentioned problem of sub-optimal efficiency of the CAD system, by not requiring the CAD system to generate and provide output to the physician in cases in which doing so is not likely to improve accuracy. Such embodiments of the present invention increase the efficiency of the CAD system itself by reducing the number of computations that the CAD system performs, namely by eliminating (relative to the prior art) computations involved in generating and providing output to the physician. Such embodiments of the present invention, therefore, reduce the number of computations required to be performed by the computer processor in each case, thereby resulting in a more efficient use of that processor.

Various Computer Aided Physician Documentation (CAPD) systems exist for understanding the context of a medical study and the content of a partially written report (also referred to herein as a “clinical note” or “note”) as it is being authored, and to annotate such a report (e.g., with measurements, findings, and diagnoses). Embodiments of the present invention may include a CAPD system which has been modified to evaluate the output of a CAD system in the context of the current note as it is being authored by the physician. For example, and as described in more detail below, embodiments of the present invention may determine whether the findings of the physician agree with or contradict the findings represented by the CAD output, and then determine whether to provide the CAD output to the physician based on the determination.

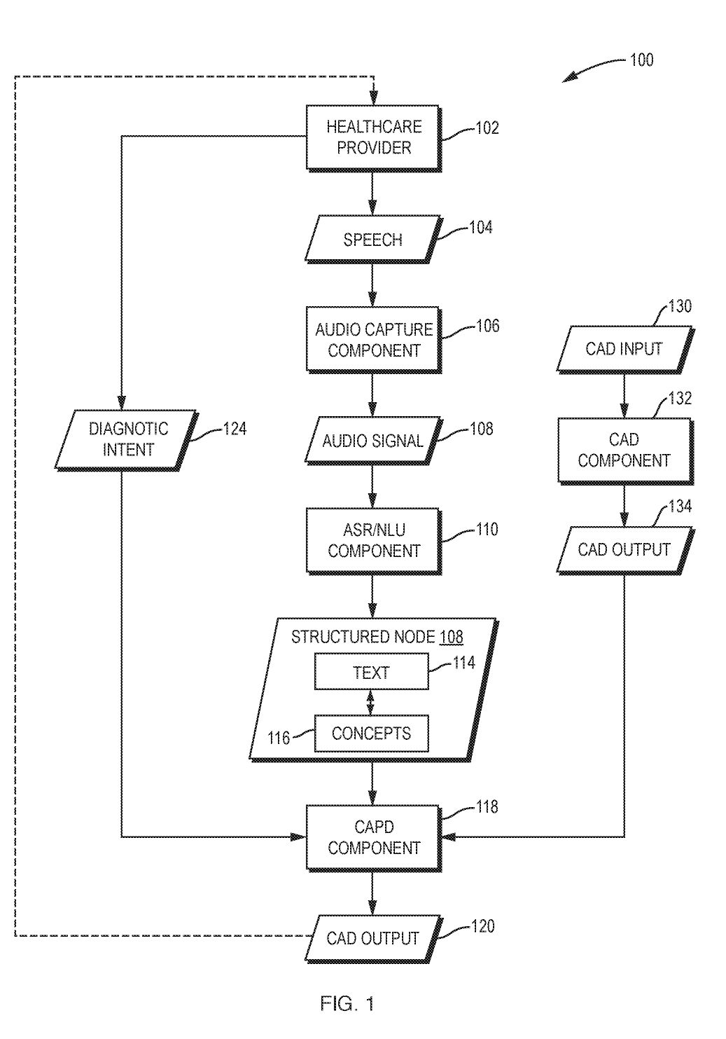

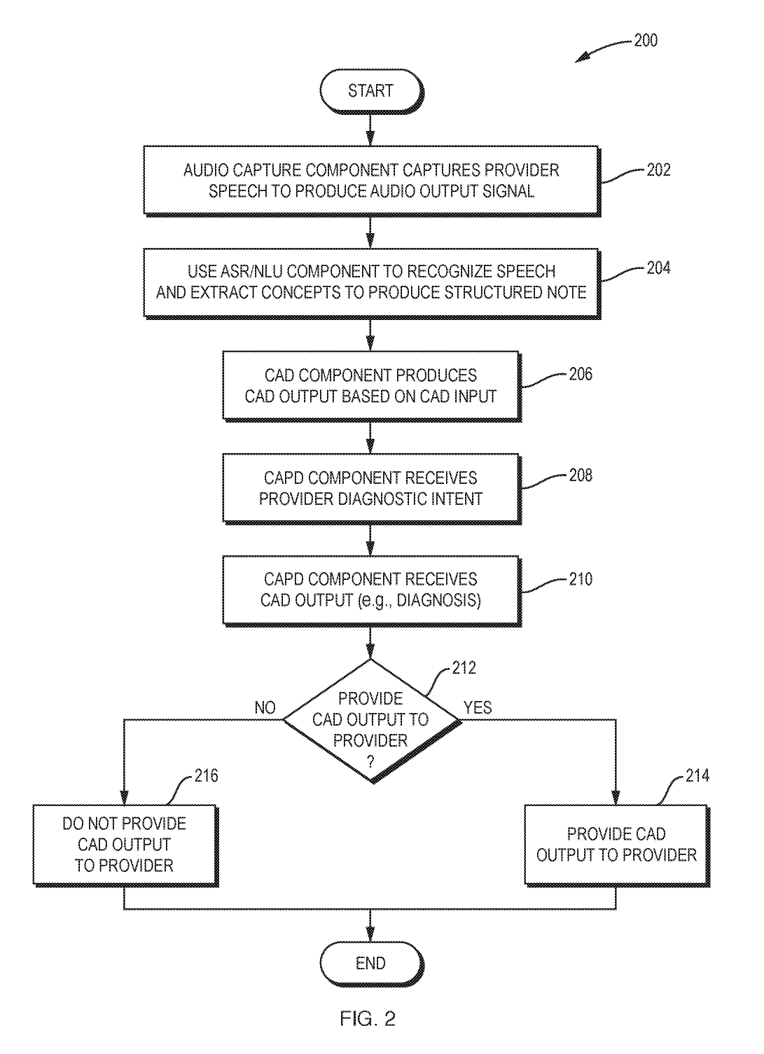

Referring to FIG. 1, a dataflow diagram is shown of a computer system 100 for automatically providing output of a CAD system to a physician only when doing so provides a benefit to the physician. Referring to FIG. 2, a flowchart is shown of a method 200 performed by the system 100 of FIG. 1 according to one embodiment of the present invention.

In the particular embodiment illustrated in FIG. 1, an audio capture component 106 captures the speech 104 of the healthcare provider 102 (e.g., a physician) during or after a patient encounter (FIG. 2, operation 202). The healthcare provider 102 may, for example, dictate a report of the patient encounter, while the patient encounter is occurring and/or after the patient encounter is completed, in which case the speech 104 may be the speech of the healthcare provider 102 during such dictation. Embodiments of the present invention, however, are not limited, however, to capturing speech that is directed at the audio capture component 106 or otherwise intended for use in creating documentation of the patient encounter. For example, the speech 104 may be natural speech of the healthcare provider 102 during the patient encounter, such as speech of the healthcare provider 102 that is part of a dialogue between the healthcare provider 102 and the patient. Regardless of the nature of the speech 104, the audio capture component 106 may capture some or all of the speech 104 and produce, based on the speech 104, an audio output signal 108 representing some or all of the speech 104. The audio capture component 106 may use any of a variety of known techniques to produce the audio output signal 108 based on the speech 104.

Although not shown in FIG. 1, the speech 104 may include not only speech of the healthcare provider 102 but also speech of one or more additional people, such as one or more additional healthcare providers (e.g., nurses) and the patient. For example, the speech 104 may include the speech of both the healthcare provider 102 and the patient as the healthcare provider 102 engages in a dialogue with the patient as part of the patient encounter.

The audio capture component 106 may be or include any of a variety of well-known audio capture components, such as microphones, which may be standalone or integrated within or otherwise connected to another device (such as a smartphone, tablet computer, laptop computer, or desktop computer).

In the particular embodiment illustrated in FIG. 1, the system 100 also includes an automatic speech recognition (ASR) and natural language understanding (NLU) component 110, which may perform automatic speech recognition and natural language understanding (also referred to as natural language processing) on the audio signal 108 to produce a structured note 112, which contains both text 114 representing some or all of the words in the audio signal 108 and concepts extracted from the audio signal 108 and/or the text 114 (FIG. 2, operation 204). The ASR/NLU 110 may, for example, perform the functions disclosed herein using any of the techniques disclosed in U.S. Pat. No. 7,584,103 B2, entitled, “Automated Extraction of Semantic Content and Generation of a Structured Document from Speech” and U.S. Pat. No. 7,716,040, entitled, “Verification of Extracted Data,” which are hereby incorporated by reference herein.

The ASR/NLU component may be implemented in any of a variety of ways, such as in one or more software programs installed and executing on one or more computers. Although the ASR/NLU component 110 is shown as a single component in FIG. 1 for ease of illustration, in practice the ASR/NLU component may be implemented in one or more components, such as components installed and executing on separate computers.

Although in the particular embodiment illustrated in FIG. 1, the structured note 108 is generated from the speech 104 of the healthcare provider 102, this is merely an example and not a limitation of the present invention. The structured note 108 may, for example, be generated based entirely or in part on input other than speech 104. In other words, the system 100 may, for example, generated the structured note 108 based on a combination of speech input 104 and non-speech input (not shown), or based entirely on non-speech input. Such non-speech input may include, for example, plain text, structured text, data in a database, data scraped from a screen image, or any combination thereof. The healthcare provider 102 may provide such non-speech input by, for example, typing such input, using discrete user interface elements (e.g., dropdown lists and checkboxes) to enter such input, or any combination thereof. Any such input may be provided to an NLU component (such as the NLU component in element 110) to create the structured note 108 in any of the ways disclosed herein.

The structured note 112, whether created based on speech input 104, non-speech input, or a combination thereof, may take any of a variety of forms, such as any one or more of the following, in any combination: a text document (e.g., word processing document), a structured document (e.g., an XML document), and a database record (e.g., a record in an Electronic Medical Record (EMR) system). Although the structured note 112 is shown as a single element in FIG. 1 for ease of illustration, in practice the structured note 112 may include one or more data structures. For example, the text 114 and the concepts 116 may be stored in distinct data structures. The structured note 112 may include data representing correspondences (e.g., links) between the text 114 and the concepts 116. For example, if the concepts 116 include a concept representing an allergy to penicillin, the structured note 112 may include data pointing to or otherwise representing text within the text 114 which represents an allergy to penicillin (e.g., “Patient has an allergy to penicillin”).

The system 100 also includes an Computer Aided Diagnostic (CAD) component 132, which receives CAD input 130 as input and processes the CAD input 130 to produce CAD output 134 (FIG. 2, operation 206). The CAD input 130 may, for example, include one or more medical images, such as one or more radiology images of a patient (e.g., ultrasound, CT, and/or MRI images). The CAD component 132 may use any of a variety of well-known techniques to produce the CAD output 134, which may include data generated automatically by the CAD component 132 and which represents one or more diagnoses of the patient based on the CAD input 130. Although not shown in FIG. 1, the CAD component 132 may receive the structured note 108 as an additional input and use the structured note 108, in combination with the CAD input 130, to generate the CAD output 134.

The system 100 may also include a Computer Aided Physician Documentation (CAPD) component 118, which may include any of a variety of existing CAPD technologies, as well as being capable of performing the functions now described. The healthcare provider 102 may provide a diagnostic intent input 124 to the CAPD component 118, which may receive the diagnostic intent input 124 as input (FIG. 2, operation 208). The healthcare provider 102 may, for example, generate and input the diagnostic intent input 124 manually, such as by dictating and/or typing the input 124.

The diagnostic intent input 124 may include any of a variety of data representing a diagnostic intent of the healthcare provider in connection with the patient. Such input 124 may, but need not, represent a diagnosis of the patient by the healthcare provider 102. The input 124 may, for example, include data representing a diagnostic intent of the healthcare provider in connection with the patient but which does not represent a diagnosis of the patient by the healthcare provider. As an example of the latter, the diagnostic intent input 124 may include a description of the healthcare provider 102's observations (findings) of the patient and also include a description of the healthcare provider 102's impressions of their observations. Such findings and impressions may indicate a diagnostic intent of the healthcare provider but not represent a diagnosis.

Although the healthcare provider 102 may generate and provide the diagnostic intent input 124 after the structured note 112 has been generated in its entirety, this is not a limitation of the present invention. The healthcare provider 102 may, for example, generate some or all of the diagnostic intent input 124 while the structured note 112 is being generated and before the entire structured note 112 has been generated, e.g., while any of one or more of the following is occurring:
while the healthcare provider 102 is providing input to the ASR/NLU component 110 (e.g., while the healthcare provider 102 is speaking to produce the speech 104 and/or while the healthcare provider 102 is generating other input to generate the structured note 108) and before the healthcare provider 102 has produced all of the input to generate the structured note 108;
while the audio capture component 106 is capturing the speech 104 and before the audio capture component 106 has captured all of the speech 104;
while the audio capture component 106 is generating the audio signal 108 and before the audio capture component 106 has generated all of the audio signal 108; and
while the ASR/NLU component 110 is generating the structured note 112 (e.g., based on the speech 104 and/or non-speech input) and before the ASR/NLU component 110 has produced all of the structured note 112.

For example, the healthcare provider 118 may provide the diagnostic intent input 124 after inputting one section of the structured note 108 (e.g., the Findings section) and before inputting another section of the structured note 108 (e.g., the Impressions section). As a result, the CAPD component 118 may receive the provider diagnosis 124 while the structured note 108 is being generated (i.e., after some, but not all, of the structured note 108 has been generated).

The CAPD component 118 may also receive the CAD output 134 (which may include data representing a diagnosis generated by the CAD component 132) as input (FIG. 2, operation 210). As is well-known to those having ordinary skill in the art, the CAD output 134 may include a variety of other data, such as prior known CAD output and a confidence level in the CAD output 134. Although the CAD component 132 may generate and provide the CAD output 134 to the CAPD component 118 after the structured note 112 has been generated in its entirety, this is not a limitation of the present invention. The CAD component 132 may, for example, generate some or all of the CAD output 134 while the structured note 112 is being generated and before the entire structured note 112 has been generated, e.g., while any of one or more of the following is occurring:
before the healthcare provider 102 begins to provide input to generate the structured note 108 (e.g., the speech 104 or non-speech input)
while the healthcare provider 102 is providing input to generate the structured note 108 (e.g., the speech 104 or non-speech input)) and before the healthcare provider 102 has produced all of the input to generate the structured note 108;
while the audio capture component 106 is capturing the speech 104 and before the audio capture component 106 has captured all of the speech 104;
while the audio capture component 106 is generating the audio signal 108 and before the audio capture component 106 has generated all of the audio signal 108; and
while the ASR/NLU component 110 is processing the audio signal 108 to produce the structured note 112 and before the ASR/NLU component 110 has produced all of the structured note 112.

The CAPD component 118 may, after receiving both the provider diagnostic intent input 124 and the CAD output 134, determine whether to provide output 120 representing some or all of the CAD output 134 to the healthcare provider 102, based on any one or more of the following, in any combination (FIG. 2, operation 212):
the provider diagnosis 124;
the CAD output 134;
a known error rate of the healthcare provider 102; and
any other data within the CAD output 134 (e.g., prior known CAD output, confidence level).

If the CAPD component 118 determines that the CAD output 134 should be provided to the provider 102, then the CAPD component 118 provides the output 120 (representing some or all of the CAPD output 134) to the provider 102 (FIG. 2, operation 214). If the CAPD component 118 determines that the CAD output 134 should not be provided to the provider 102, then the CAPD component 118 does not provide some or all of the output 120 (representing some or all of the CAPD output 134) to the provider 102 (FIG. 2, operation 216); the CAPD component 118 may not even generate any of the output 120 in this case.

The CAPD component 118 may use any of a variety of techniques to determine whether to provide the CAD output 134 to the healthcare provider 102. In general, if the CAD output 134 agrees with the diagnostic intent input 124, then the system 100 provides the CAD output 134 to the healthcare provider 102; otherwise the system 100 does not provide the CAD output 134 to the healthcare provider 102. A refinement of this general approach is that the system 100 may not provide the CAD output 134 to the healthcare provider 102 in response to determining that the diagnostic intent input 124 includes a finding that the CAD output 134 does not include, but otherwise provide the CAD output 134 to the healthcare provider 102.

More specifically, for example, the CAPD component 118 may determine whether the provider diagnostic intent 124 is the same as or otherwise consistent with (e.g., contains findings that are consistent with) the diagnosis represented by the CAD output 134. Then the CAPD component 118 may act as follows:
If the provider diagnostic intent 124 is the same as or otherwise consistent with the diagnosis represented by the CAD output 134, then the CAPD component 118 may not provide the CAD output 134 to the provider 102. This is because providing a redundant CAD diagnosis 134 to the healthcare provider 102, whether that diagnosis 134 is correct or incorrect, does not provide the healthcare provider 102 with information that is useful to the healthcare provider 102 in evaluating the correctness of the healthcare provider 102's own diagnostic intent input 124.
If the CAD output 134 classifies a case as not containing a relevant finding, but the provider diagnosis 124 classifies the case as containing a relevant finding, then the CAPD component 118 may (depending on the relative cost of false positives and false negatives) not provide the full CAD output 134 to the healthcare provider 102, and instead only provide to the healthcare provider 102 information (from the CAD output 134) about those cases in which the healthcare provider 102 (in the provider diagnosis 124) classified a study as “normal” but which the CAD output 134 identified as “abnormal.”
Alternatively, if the provider diagnosis 124 disagrees with the diagnosis represented by the CAD output 134, then the CAPD component 118 may not provide any of the CAD output 134 to the healthcare provider 102, but route the CAD input 130 (e.g., radiology image) to a human reviewer (not shown) other than the healthcare provider 102 to increase reading accuracy.
The CAPD component 118 may track agreement of multiple provider diagnostic intent inputs (including the provider diagnostic intent input 124) with multiple CAD outputs (including the CAD output 134) over time and aggregate information about the frequency of agreement/disagreement between such human and computer-generated diagnoses, and use such aggregate information to estimate the accuracy of the CAD component 132 (such as the user-dependent accuracy and/or global accuracy of the CAD component 132) by either explicating eliciting and receiving user feedback on the accuracy of the CAD component 132 or by analyzing the agreement of the physician's diagnostic intents (e.g., diagnostic intent 124) with the CAD output (e.g., CAD output 134). The system 100 may display, to the healthcare provider 102, information about the percentage agreement in order to help the healthcare provider to judge the reliability of the CAD output 134.

Embodiments of the present invention have a variety of advantages. For example, the system 100 and method 200 reduce the amount of time required by the healthcare provider 102 to review CAD-generated diagnoses, by only providing those diagnoses as output to the healthcare provider 102 in cases in which providing such diagnoses is likely to improve the accuracy of the healthcare provider 102's diagnosis. In typical uses cases this can reduce the amount of unnecessary effort required by the healthcare provider 102 by a significant amount, without any reduction in diagnosis accuracy, and possibly with an increase in diagnosis accuracy as a result of increasing the healthcare provider 102's confidence in the system 100 and reducing the healthcare provider 102's workload, thereby enabling the healthcare provider 102 to focus more carefully on reviewing the relatively small number of CAD diagnoses that are likely to be helpful in improving the accuracy of the healthcare provider 102's diagnosis.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention use computerized automatic speech recognition, natural language understanding, computer-aided diagnostic, and computer-aided physician documentation components to automatically recognize and understand speech, to automatically generate diagnoses, and to automatically understand the context of a clinical note. Such components are inherently computer-implemented and provide a technical solution to the technical problem of automatically generating documents based on speech.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

What is claimed is:

1. A method performed by at least one computer processor executing computer program instructions stored on at least one non-transitory computer-readable medium, the method comprising:
(A) using a computer aided diagnostic (CAD) system to generate a CAD diagnosis automatically based on a medical image;
(B) receiving the CAD diagnosis from the CAD system;
(C) receiving input representing a diagnostic intent of a human user;
(D) determining, based on the CAD diagnosis and the diagnostic intent, whether to provide the CAD diagnosis to the human user; and
(E) only providing the CAD diagnosis to the human user if it is determined that the CAD diagnosis should be provided to the human user.

2. The method of claim 1, wherein (D) comprises determining whether the CAD diagnosis agrees with the diagnostic intent of the human user, and wherein (E) comprises only providing the CAD diagnosis to the human user if it is determined that the CAD diagnosis agrees with the diagnostic intent of the human user.

3. The method of claim 1, wherein (D) comprises determining whether the diagnostic intent of the human user includes a finding that the CAD diagnosis does not include, and wherein (E) comprises not providing the CAD diagnosis to the human user if it is determined that the diagnostic intent of the human user includes a finding that the CAD diagnosis does not include, and otherwise providing the CAD diagnosis to the human user.

4. The method of claim 1, wherein (B) further comprises receiving prior output from the CAD system, and wherein (D) comprises determining, based on the CAD diagnosis, the prior output, and the diagnostic intent, whether to provide the CAD diagnosis to the human user.

5. The method of claim 1, wherein (B) further comprises receiving a confidence level in the CAD diagnosis, and wherein (D) comprises determining, based on the CAD diagnosis, the confidence level in the CAD diagnosis, and the diagnostic intent, whether to provide the CAD diagnosis to the human user.

6. The method of claim 1, wherein (B) further comprises receiving a known error rate of the human user, and wherein (D) comprises determining, based on the CAD diagnosis, the known error rate of the human user, and the diagnostic intent, whether to provide the CAD diagnosis to the human user.

7. The method of claim 1, wherein (C) comprises:
(C) (1) using an audio capture component to capture speech of the human user; and
(C) (2) using an automatic speech recognition (ASR) component to perform ASR on the speech of the human user and thereby to produce text representing the speech; and
wherein the input representing the diagnostic intent comprises the text.

8. The method of claim 7, wherein the input representing the diagnostic intent comprises some, but not all, of the text, and wherein (D) is performed before the ASR component has produced all of the text.

9. The method of claim 1, wherein (C) comprises:
(C) (1) using an audio capture component to capture speech of the human user; and
(C) (2) using a natural language understanding (NLU) component to perform NLU on the speech of the human user and thereby to produce data representing concepts in the speech; and
wherein the input representing the data representing the concepts.

10. The method of claim 1, wherein the input representing the diagnostic intent of the human user comprises input representing a diagnosis.

11. A system comprising at least one non-transitory computer-readable medium having computer program instructions stored thereon, wherein the computer program instructions are executable by at least one computer processor to execute a method, the method comprising:
(A) using a computer aided diagnostic (CAD) system to generate a CAD diagnosis automatically based on a medical image;
(B) receiving the CAD diagnosis from the CAD system;
(C) receiving input representing a diagnostic intent of a human user;
(D) determining, based on the CAD diagnosis and the diagnostic intent, whether to provide the CAD diagnosis to the human user; and
(E) only providing the CAD diagnosis to the human user if it is determined that the CAD diagnosis should be provided to the human user.

12. The system of claim 11, wherein (D) comprises determining whether the CAD diagnosis agrees with the diagnostic intent of the human user, and wherein (E) comprises only providing the CAD diagnosis to the human user if it is determined that the CAD diagnosis agrees with the diagnostic intent of the human user.

13. The system of claim 11, wherein (D) comprises determining whether the diagnostic intent of the human user includes a finding that the CAD diagnosis does not include, and wherein (E) comprises not providing the CAD diagnosis to the human user if it is determined that the diagnostic intent of the human user includes a finding that the CAD diagnosis does not include, and otherwise providing the CAD diagnosis to the human user.

14. The system of claim 11, wherein (B) further comprises receiving prior output from the CAD system, and wherein (D) comprises determining, based on the CAD diagnosis, the prior output, and the diagnostic intent, whether to provide the CAD diagnosis to the human user.

15. The system of claim 11, wherein (B) further comprises receiving a confidence level in the CAD diagnosis, and wherein (D) comprises determining, based on the CAD diagnosis, the confidence level in the CAD diagnosis, and the diagnostic intent, whether to provide the CAD diagnosis to the human user.

16. The system of claim 11, wherein (B) further comprises receiving a known error rate of the human user, and wherein (D) comprises determining, based on the CAD diagnosis, the known error rate of the human user, and the diagnostic intent, whether to provide the CAD diagnosis to the human user.

17. The system of claim 11, wherein (C) comprises:
(C) (1) using an audio capture component to capture speech of the human user; and
(C) (2) using an automatic speech recognition (ASR) component to perform ASR on the speech of the human user and thereby to produce text representing the speech; and
wherein the input representing the diagnostic intent comprises the text.

18. The system of claim 17, wherein the input representing the diagnostic intent comprises some, but not all, of the text, and wherein (D) is performed before the ASR component has produced all of the text.

19. The system of claim 11, wherein (C) comprises:
(C) (1) using an audio capture component to capture speech of the human user; and
(C) (2) using a natural language understanding (NLU) component to perform NLU on the speech of the human user and thereby to produce data representing concepts in the speech; and
wherein the input representing the data representing the concepts.

20. The system of claim 11, wherein the input representing the diagnostic intent of the human user comprises input representing a diagnosis.
